(19) United States
(12) Patent Application Publication
(10) Pub. No.: US 2026/0283470 A1
(43) Pub. Date: Sep. 24, 2026

(54) MEDICAL IMAGING SYSTEM AND METHOD FOR OPERATING A MEDICAL IMAGING SYSTEM

(71) Applicant: KARL STORZ SE & Co. KG, Tuttlingen (DE)

(72) Inventors: Werner GÖBEL, Tuttlingen (DE); Benedikt KÖHLER, Tuttlingen (DE)

(21) Appl. No.: 19/141,142

(22) PCT Filed: Dec. 19, 2023
(86) PCT No.: PCT/EP2023/000063

§ 371 Date: Jun. 19, 2025

(30) Foreign Application Priority Data
Dec. 30, 2022 (DE) 10 2022 135 045.2
Dec. 30, 2022 (DE) 10 2022 135 046.0

(51) Int. Cl. A61B 5/00; A61B 90/90

(52) U.S. Cl. CPC A61B 5/0075; A61B 90/90; A61B 2560/0252; A61B 2576/00

(57) ABSTRACT

The invention provides a medical imaging system, in particular for multispectral imaging, and a method for operating such an imaging system. The imaging system (100) comprises:
a light source (110), which is designed to irradiate an object (10) with light of different wavelengths, according to a current wavelength spectrum of a plurality of possible wavelength spectra of the light source (110), wherein the light source (110) is assigned an individual identification code, IIC,
a first memory (111), which is designed to store the individual identification code, IIC, of the light source (110),
a second memory (112), which is designed to store spectrum information concerning possible wavelength spectra of the light source (110),
a camera (130), which is designed to generate image data of the object (10) with light reflected or emitted from the object (10), based on the light according to the current wavelength spectrum,
a controller (140), which is designed to adapt the generation of image data by the camera (130) using the individual identification code, IIC, of the light source (110) and the spectrum information.

### FIELD OF THE INVENTION

The present invention relates to a medical imaging system, in particular for multispectral imaging. Furthermore, the invention relates to a method for operating such a medical imaging system, a computer program product, a data storage medium and a data stream.

### TECHNICAL BACKGROUND

During medical examinations, a doctor often has to distinguish between different types of organic tissue, not only based on the type of tissue but also on the current condition of the tissue. For example, in the case of anastomosis of blood vessels or intestinal sections, it is essential to know which tissue sections are supplied with oxygen (oxygenated) and which are not (deoxygenated), in order to, for instance, avoid suturing non-perfused tissue to perfused tissue, which could lead to necrosis or other complications.

Methods for analyzing tissue based on its electromagnetic spectrum are known in the prior art. U.S. Pat. No. 10,156,518 B2 describes a method using a “spectral angle mapper,” wherein a similarity of an intensity spectrum of a pixel to a reference spectrum is determined using angles in a parameter space.

In multispectral imaging, light intensities at a variety of different wavelengths are captured and analyzed in order to determine various medical or physiological parameters, for example oxygen saturation or the like. For this purpose, a light source is used which emits light that is then partially reflected and partially absorbed by the object under examination (absorption spectroscopy). However, due to manufacturing tolerances and other influences, the wavelength spectrum of the emitted light may vary depending on the device and situation. This can also cause fluctuations in the captured absorption spectrum, which can make accurate analysis of the object under examination difficult.

It is known that a manual white balance of a light source is advantageous. However, this is often cumbersome, in particular with multispectral light sources and sensors. Furthermore, a white balance using an external reference object cannot be performed in every situation.

U.S. Pat. No. 8,300,906 B2 describes a surgical system with a surgical lamp and a camera, wherein a white balance of a visual representation on a monitor is adapted based on color temperature information of the surgical lamp. This solution requires a color temperature sensor, which introduces additional uncertainties into the system. In particular, both the function of the surgical lamp and the color temperature sensor may decrease over the service life of the surgical system or as the operating temperature of the surgical system increases.

JP 2003/334159 A describes a camera in which adaptation values for different light source types are stored. Color correction of images generated by the camera is performed using the stored adaptation values. This teaching leads to relatively rough settings since even light sources of the same type can sometimes differ significantly in their color spectrum.

DE 195 29 367 A1 describes a device for color correction in images from a video camera. After a white balance, a color correction can be set using a rotary encoder using a table stored in a memory of the video camera. The setting remains until a new white balance is performed. Setting the color correction using the rotary encoder is cumbersome and not very precise.

U.S. Pat. No. 8,427,534 B2 describes a processor for an electronic endoscope. The processor comprises a light source and receives an image generated by a videoscope which is connected to the endoscope. Based on this, the processor can determine whether color adaptation data which is used by the videoscope for color adaptation is adapted to the employed light source. If this is not the case, color conversion data can be captured which is used to apply a color conversion to the image data transmitted by the videoscope. The described process is relatively cumbersome and also only affects the image data after it has been generated.

Accordingly, it is an object of the present invention to provide an improved medical imaging system and an improved method for operating such a medical imaging system. In particular, the quality of the generated image data is to be improved.

This object is achieved by the subject matter of the independent claims.

According to a first aspect, a medical imaging system, in particular for multispectral imaging, is provided, the imaging system comprising: a light source which is designed to irradiate an object with light of different wavelengths according to a current wavelength spectrum of the light source, wherein the light source is assigned an individual identification code, IIC,
a first memory which is designed to store the individual identification code of the light source, a second memory which is designed to store spectrum information concerning possible wavelength spectra of the light source,
a camera which is designed to generate image data of the object with light reflected or emitted from the object based on the light according to the current wavelength spectrum,
a controller, which is designed to adapt the generation of image data by (or using) the camera using the individual identification code of the light source and the spectrum information.

An underlying idea of the present invention is that a more precise knowledge of the emitted light spectrum is linked to a specific light source, i.e. to a specific individual identification code, IIC, and is particularly easily accessible via this. Multispectral imaging and/or absorption spectroscopy can thus be carried out with particular accuracy.

In the foregoing and following, terms are sometimes abbreviated with acronyms, for instance “IIC” for “individual identification code.” Typically, the full form is used, followed by the corresponding acronym. However, in some cases, to improve readability, only the acronym will be used, while in other cases the acronym will be omitted. In every case, the acronym and the full form shall be understood to have the same meaning.

The spectrum information associated with the light source and thus with the individual identification code, IIC, can be generated, for example, during the production of the light source or subsequently by measurements (e.g., on a test bench). Such test benches typically have high-precision measuring instruments. The spectrum information generated in this way is generally more comprehensive and accurate than, for example, color temperature information recorded during operation of the light source.

It is understood that the current wavelength spectrum is one of the possible wavelength spectra of the light source. In the present case, possible wavelength spectra are primarily those that are not deliberately selected by a certain setting, but rather those that result from external or internal parameters or their changes, e.g., due to aging processes or changes in operating temperature. In other words, “possible” wavelength spectra are not necessarily those that the light source can optionally emit at a certain point in time, but in particular those that the light source may only be capable of emitting in certain circumstances in the future (aging) and/or under special circumstances (operating temperature).

Corresponding information as to under which circumstances which wavelength spectrum is possible or mandatory can be contained in the spectrum information, preferably in each case together with the corresponding wavelength spectrum (e.g., an associated spectral intensity distribution) or adaptation data with which, e.g., a standard wavelength spectrum can be corrected into the corresponding wavelength spectrum.

This means that the spectrum information can, for example, be a set of wavelength spectra, in each case with corresponding information concerning the external circumstances and/or internal states of the light source under which this wavelength spectrum is present. The spectrum information can also be or have corresponding adaptation data which indicate how a standard wavelength spectrum—general or light source-dependent—is to be adapted, again depending on external circumstances and/or internal states of the light source. For example, it can be saved in the spectral information on a light source that wavelength A is present with intensity C1 under condition B=B1. Furthermore, it can be saved in the spectral information that, on the one hand, wavelength A is present with intensity C2 under condition B=B2 (set of wavelength spectra), or, on the other hand, that under condition B=B2, the intensity C1 of wavelength A is increased or decreased by (C2−C1) (adaptation data). It does not matter whether a wavelength spectrum is specified by indicating all intensity values or the like, or by indicating the adaptation data.

The invention is in particular intended for use in multispectral imaging. In particular, “multispectral” means that each wavelength profile has intensity information for at least two different electromagnetic wavelengths. The wavelength profile can therefore also be described as a multispectral intensity profile. In multispectral imaging, intensity distributions are measured for a plurality of defined electromagnetic wavelengths, preferably for each wavelength that is part of the wavelength spectrum (or the multispectral intensity profile). Based on the measured intensity distributions, not only can images in the visible range be output, but additional medical or physiological parameters can also be determined, e.g. by an evaluation module, for example an oxygen saturation of a body region of a patient, a hemoglobin content, a water content or a perfusion, for example within the scope of near-infrared perfusion angiography.

Multispectral wavelength profiles also include so-called hyperspectral intensity profiles which provide a continuous description of the intensity as a function of the electromagnetic wavelength (within a predetermined definition range). By discretely reading out individual values (or by interval-based integration and averaging), a discrete multispectral intensity profile can be generated from any hyperspectral intensity profile. Conversely, a hyperspectral intensity profile can also be generated by interpolation from a discrete multispectral intensity profile. However, it is preferred to use discrete intensity profiles for ease of handling, i.e. to automatically correspondingly convert hyperspectral intensity profiles, if available, into discrete intensity profiles.

The controller is preferably connected directly or indirectly to the camera, the light source and the first and/or the second memory via signal lines. However, some, a plurality or all signal transmissions of data signals can also be carried out via a wireless interface, e.g. Bluetooth, ZigBee, LoraWAN, Ethernet or the like.

Adapting the generation of image data using the camera can involve correcting the image data, preferably by removing or compensating for effects that arise due to the wavelength spectrum used to irradiate the object not being spectrally uniform, i.e., different wavelengths being emitted at different intensities. For example, the reflected light intensity of a (any) certain wavelength captured by the camera can be normalized to the light intensity with which light of such wavelength was originally emitted by the light source. A data cube of image data, which contains intensity data for each captured wavelength for each sensor pixel of the camera, can thus, for example, be generated in such a way that it only contains data between 0 and 1.

By assigning the spectrum information to specific light sources or individual identification codes, adapting the generation of image data can be carried out quickly and with little effort.

The controller (or control device) can be any device that is designed and configured for digital computing, in particular for running software, an application or an algorithm. The controller may, for example, comprise at least one processor unit (e.g., at least one CPU), at least one graphics processor unit (e.g., at least one GPU), at least one field-programmable gate array, FPGA, and/or at least one application-specific integrated circuit, ASIC, and/or any combination of the aforementioned elements. The controller can also be referred to as a camera control unit or CCU.

The controller can also comprise a working memory and/or a non-volatile data memory that are operatively linked to each other and/or to some or all of the aforementioned elements. The controller can be implemented partially or entirely in a local unit (e.g., a personal computer, PC, laptop, notebook, or the like) and/or partially or entirely in a distributed system, e.g., a cloud computing platform or a server arranged spatially apart.

When different memories (first memory, second memory, etc.) are mentioned within the scope of the present invention and disclosure, this is initially a functional distinction to improve understanding. It is understood that a plurality or all of these memories can be implemented together in one or more hardware storage devices (e.g., a solid-state memory). In the present case, hardware storage devices can be arranged in particular in the light source, in the camera and/or in the controller. One or more memories can also be implemented in a cloud database or on a cloud server. For the first memory which stores the individual identification code, IIC, of the light source, it is particularly advantageous if this is arranged in the assigned light source itself. Any memory can also be referred to as a “data memory.”

Within the scope of the present invention and disclosure, data transmissions are often described, i.e., one entity (e.g., the controller) receives data from another entity (e.g., the light source) or vice versa. It is understood that this can always be carried out using methods known in the prior art. For example, data transmission can be wired or wireless. In addition, data transmission can be carried out using the push method or the pull method. In the push method, an entity (e.g., the controller) receives the data as soon as they are generated (e.g., at the light source). In the pull method, one entity (e.g., the controller) requests the data from another entity (e.g., the light source) and then obtains it.

Furthermore, data transmission can be performed regularly, regardless of whether the data are requested and/or new data are available. One or more of these variants can also be combined. It is understood that when designing a specific embodiment, a person skilled in the art will always select the appropriate form of data transmission based on the information contained therein. Concretely described forms of data transmission can thus always be replaced by others.

Further preferred embodiments, variants and developments of embodiments can be found in the dependent patent claims as well as in the description with reference to the figures.

According to some preferred embodiments, variants or further developments of embodiments, the controller is designed to provide correction data from or based on the individual identification code, IIC, the light source and the spectrum information. In other words, the spectrum information can contain correction data or make their generation possible. The correction data can be used to adapt an algorithm for generating the image data in the camera, e.g., to provide it with changed parameters.

The medical imaging system can further comprise a third memory that is designed to store the correction data. The controller can be designed to adapt the generation of image data by the camera based on the stored correction data. The third memory can also be designed to store the spectrum information (in whole or in part). Providing the correction data can comprise reading out from the spectrum information, calculating, selecting, etc. using the individual identification code, IIC.

For example, the first memory for storing the individual identification code, IIC, as well as the second memory for storing the spectrum information can be arranged in the light source, while the third memory is arranged in the controller. In this way, the correction data can be generated and stored in the controller as required. As long as the correction data are up to date, no further data exchange is necessary. Whether they are up to date can be ascertained by the controller using the individual identification code, IIC.

In medical imaging systems, data exchange between the light source and the controller is often carried out as part of a “handshake,” for instance to check whether the light source is compatible with the controller and/or is approved for use with the controller. Thus, the individual identification code, IIC, can advantageously be transmitted from the light source to the controller via the same interface. This interface can be a wireless interface, wherein wired interfaces are often preferred.

According to some preferred embodiments, variants or further developments of embodiments, the medical imaging system comprises a temperature sensor device which is designed to capture an operating temperature value of the light source during operation and to transmit the captured operating temperature value to the controller. The controller can be designed to additionally adapt the generation of the image data based on the operating temperature value.

The spectrum information can have information that allocates a wavelength spectrum of the light source in each case to a plurality of operating temperature values (or even to each operating temperature value) for the light source with the associated individual identification code, IIC. The controller can be designed to select a wavelength spectrum assigned to the captured operating temperature value based on the spectrum information and to adapt the generation of the image data based on the selected wavelength spectrum. As already mentioned, the allocation of wavelength spectra to operating temperature values can be carried out either directly via intensity data of the wavelength spectra or via adaptation data with respect to a standard wavelength spectrum. The operating temperature values can also be present as absolute values or as differential values from a standard operating temperature value. A large number of equivalent implementations is conceivable.

The spectrum information may, for example, contain a table for each individual identification code, IIC, which contains a wavelength spectrum for each operating temperature value. Generating image data can then be adapted depending on these wavelength spectra. However, the spectrum information can also have correction data that depend on the operating temperature value. For example, a formula with parameter variables can be saved in the controller, and the spectrum information can allocate a set of parameters for the parameter variables for each individual identification code, IIC, of a plurality of operating temperature values. In this way, the controller can obtain or calculate correction data (e.g., sets of parameters) which can be stored, for example, in the third memory mentioned above.

According to some preferred embodiments, variants or further developments of embodiments, the temperature sensor is designed to capture the operating temperature value of the light source on request or at predetermined time intervals. The light source, in particular the temperature sensor device, can further be designed to communicate the captured operating temperature value to the controller, which is designed to check whether the captured operating temperature value is assigned to the currently selected lighting spectrum. In other words, the controller can check whether generating the image data is currently being performed based on a wavelength spectrum with which the object is currently irradiated due to the operating temperature value of the light source.

The controller can further be designed, in the event of an unsuitable assignment (in particular due to a change in the operating temperature value, for example due to increasing heating of the light source during operation), to select a different wavelength spectrum that is assigned to the captured operating temperature value. Depending on how the spectrum information is designed, the wavelength spectrum can be selected by selecting from a list of wavelength spectra, by adapting a standard wavelength spectrum using adaptation data, or the like. The actual adaptation of the generation of the image data can be performed by a program that obtains a wavelength spectrum as input and controls the generation of the image data based on this, for example by setting parameters based on this.

In other words, the generation of image data can be adapted to the current operating temperature value of the light source. For this purpose, it is advantageous if the spectrum information, which assigns the possible wavelength spectra of the light source to the operating temperature values of the light source, is stored in a memory, and this memory is arranged in the controller. Thus, it is sufficient to obtain the operating temperature value from the light source (for instance, using the push method, the pull method or regularly) and, if applicable, to select a different wavelength spectrum from or based on the spectrum information.

Alternatively, however, it can also be provided that the second memory is arranged in the light source and, when the operating temperature value changes, all the spectrum information is transmitted to the controller. Optionally, instead of the entire spectrum information, spectrum information that is precisely adapted to the changed operating temperature value (in particular the assigned wavelength spectrum or parameters that allow its calculation) can be transmitted.

The light source can also contain a control logic that, in each case based on the currently measured operating temperature value, selects a corresponding item of spectrum information, in particular an assigned wavelength spectrum (or parameters for its calculation) and transmits it to the controller. In this variant, the controller has no knowledge of the changed operating temperature value, but only, if applicable, that the wavelength spectrum to be used for adapting the image data has changed.

Analogous to taking into account the operating temperature value, further time-dependent properties of the particular light source can also be taken into account. If, for example, a change in the wavelength spectrum of the light source as a function of elapsed service life or elapsed usage time of the light source is known and its dependency is saved in the spectrum information, this change can also be taken into account when generating the image data. For this purpose, it is advantageous if a fourth memory in the light source stores an elapsed service life or elapsed usage time (in each case provided, e.g., by the controller during use). Analogous to what was described with reference to the operating temperature value, the controller can thus always be provided with a wavelength spectrum adapted to the current service life/usage time of the light source.

According to a second aspect, the present invention provides a medical imaging system, in particular for multispectral imaging, comprising:
a light source which is designed to irradiate an object with light of different wavelengths according to a current wavelength spectrum of the light source which depends at least on the current operating temperature of the light source,
a memory which is designed to store spectrum information concerning possible wavelength spectra depending on the operating temperature of the light source,
a temperature sensor device which is designed to capture an operating temperature value of the light source during operation,
a camera which is designed to generate image data of the object with light reflected or emitted from the object based on the light according to the current wavelength spectrum of the light source,
a controller which is designed to adapt the generation of image data by the camera based on (or using) the spectrum information and the captured operating temperature value. The adaptation can be carried out using both the spectrum information as well as the captured operating temperature value, but it can also be carried out using spectrum information that has already been selected or adapted based on the operating temperature value, and/or the like.

According to some preferred embodiments, variants or further developments of embodiments, the spectrum information comprises information that allocates in each case a wavelength spectrum of the light source to a plurality of operating temperature values (or preferably to each operating temperature value or at least to each possible operating temperature value), and wherein the controller is designed to select a wavelength spectrum assigned to the captured operating temperature value based on the spectrum information and to adapt the generation of the image data based on the selected wavelength spectrum. As described above, the allocation of wavelength spectra can be carried out in different ways, for example directly by specifying the intensity spectra, or using adaptation data.

The preferred embodiments, variants or further developments described below, which relate to medical imaging systems, can equally relate to embodiments of the first as well as the second aspect of the present invention.

According to some preferred embodiments, variants or further developments of embodiments, the temperature sensor device is designed to capture the operating temperature value of the light source on request and/or at predetermined time intervals and to communicate it to the controller. In particular, the controller checks whether the captured operating temperature value is assigned to the selected wavelength spectrum and, in the event of an unsuitable assignment due to a change in the operating temperature value, selects another wavelength spectrum that is assigned to the captured operating temperature value. Generating the image data is advantageously based on the currently selected wavelength spectrum that is correctly assigned to the current operating temperature value.

According to some preferred embodiments, variants or further developments of embodiments, the controller is designed to check the or an individual identification code, IIC, of the light source to see whether it is noted (or, in modern parlance, “flagged”) as known to the controller and, if this is not the case, to obtain the spectrum information of the light source (in particular from the light source), store it in a fifth memory in the controller and note the IIC of the light source as known (to the controller). Thus, data transmission between the light source and the controller can initially be limited to the individual identification code, IIC, and only if needed, i.e., if an unknown light source has been connected, can a data transmission of the spectral information (either in whole or in part) then be carried out. The noting can be carried out by storing known individual identification codes, IICs, in a sixth memory of the controller. A check of whether an IIC is known can thus consist of checking whether the IIC is stored in the sixth memory.

In some embodiments, the individual identification code, IIC, of a light source may be replaced by a type identification code, TIC. This means that instead of an individual adaptation for each light source, only one individual adaptation is performed for each type of light source. This can be in particular important if other parameters that are taken into account such as the operating temperature value or the operating time are significantly more important than variations between individual light sources of the same type.

In various embodiments, when an individual identification code (IIC) is noted from “known” as back to “unknown” can be configured differently. For example, it can be provided that only the IIC of the currently connected light source is to be noted as “known,” i.e. with each change, the spectrum information is reloaded, e.g. from the light source into the fifth memory.

Alternatively, it can be provided that the fifth memory stores the obtained spectrum information for each known IIC (i.e., for each already known light source) (first-in, first-out in case of memory overflow). Thus, if a new light source is newly connected to the medical imaging system as part of the “handshake” between the light source and the controller, it can first be checked whether this light source is known, i.e. whether the individual identification code, IIC, stored in its first memory is known to the controller (e.g., stored in the sixth memory). If this is the case, the associated spectrum information (e.g., a wavelength spectrum) can be read out from the fifth memory. If this is not the case, the spectrum information of the previously unknown light source can be obtained as described, and the light source can then be noted as “known.”

According to some preferred embodiments, variants or further developments of embodiments, adapting (or, here: correcting) the generation of the image data comprises a white balance. The white balance can be performed in the visible wavelength range and/or in a wavelength range invisible to humans, which is in particular advantageous for multispectral imaging. Correction data for performing a white balance may also be referred to as white balance data.

According to some preferred embodiments, variants or further developments of embodiments, adapting (or, here: correcting) the generation of image data comprises a color correction. Color correction can be carried out in the visible wavelength range and/or in a wavelength range invisible to humans, which is in particular advantageous in multispectral imaging.

According to some preferred embodiments, variants or further developments of embodiments, the camera is designed to record spectra. The image data can comprise spectral data, in particular spectral intensity distributions. In contrast to monochrome data, such image data allows for more diverse analysis options.

According to some preferred embodiments, variants or further developments of embodiments, the light source is designed to irradiate the object with light in visible wavelengths and in at least one invisible wavelength. The camera is advantageously designed to generate image data from light in visible and non-visible wavelengths.

According to some preferred embodiments, variants or further developments of embodiments, the medical imaging system comprises an evaluation module that evaluates the image data generated in an adapted manner for the purpose of determining physiological tissue properties, preferably for the purpose of a medical diagnosis. The evaluation module can, for example, perform a pixel-by-pixel evaluation of the image data and determine a medical or physiological parameter for a selection of pixels, or all pixels, such as an oxygen saturation (StO2), a hemoglobin content, a water content, or a perfusion value. Due to the highly accurate spectrum information, above all specific to each individual light source, these parameters can be determined with particular accuracy and thus make improved diagnoses possible. Since such analyses and evaluations are carried out in particular by recording spectra when illuminated by the light source, the current wavelength spectrum of the light source directly affects the determination of the physiological tissue properties and can distort them.

According to a third aspect, the invention provides a method for operating a medical imaging system (preferably according to the first or second aspect, particularly preferably according to the first aspect), comprising the steps of:
Providing spectrum information concerning possible wavelength spectra of a light source, e.g. in a memory of the light source;
Providing an individual identification code, IIC, of the light source;
Irradiating, by the light source, an object with light of different wavelengths according to a current wavelength spectrum of the light source;
Generating image data of the object with light reflected or emitted from the object based on the light according to the current wavelength spectrum, wherein generating the image data is adapted based on (or using) the individual identification code of the light source and the spectrum information.

According to some preferred embodiments, variants or further developments of embodiments, the method according to the third aspect comprises the further step of: Capturing an operating temperature value of the light source (in particular during irradiation), wherein adapting the generation of the image data is additionally carried out based on the captured operating temperature value. For example, the method further comprises the following step:
Selecting or calculating (e.g., by means of adaptation data) a wavelength spectrum that is assigned to the light source with the IIC for the captured operating temperature value based on the spectrum information,
wherein adapting the generation of the image data is carried out using the selected or calculated wavelength spectrum.

According to a fourth aspect, the invention provides a method for operating a medical imaging system (preferably according to the first or second aspect, particularly preferably according to the second aspect of the invention), comprising the steps of:
Providing spectrum information concerning possible wavelength spectra of a light source, which depends at least on the current operating temperature of the light source;
Irradiating, by the light source, an object with light of different wavelengths according to a current wavelength spectrum of the light source;
Capturing a current operating temperature value of the light source, in particular during irradiation; and
Generating image data of the object with light reflected or emitted by the object based on the light according to the current wavelength spectrum, wherein generating the image data is adapted based on the captured current operating temperature value of the light source and the spectrum information.

According to some preferred embodiments, variants or further developments of embodiments, the method further comprises the step of:

Providing an individual identification code, IIC, or a type identification code, TIC, of the light source, wherein generating the image data is additionally adapted based on (or using) the individual identification code of the light source or the TIC.

The method according to the third or fourth aspect can be performed in particular with the medical imaging system according to the first or second aspect, but also independently thereof. Accordingly, all options, modifications and variants described with reference to the medical imaging system according to the first or second aspect of the invention are also applicable to the method according to the third or fourth aspect and vice versa.

The method according to embodiments according to the third or fourth aspect can also comprise generating the spectrum information. For example, after the completion of production of a light source, a series of tests can be performed in which the wavelength spectrum of the light source is measured depending on the operating temperature value. The results can be stored as spectrum information, for example in a look-up table in a memory in the light source. For example, light intensities can be measured at a distance of 5 nanometers in a wavelength spectrum of the light source from 500 nanometers to 1000 nanometers. The dependence on the operating temperature value can be measured, for example, in steps with a width in the range of 1° C. to 5° C. An exemplary wavelength spectrum can comprise 101 light intensity values (from 500 to 1000 nanometers in 5-nanometer steps). Associated spectrum information, which assigns a wavelength spectrum to each measured operating temperature value, can thus, if, for example, 30 operating temperature value ranges are defined, be stored in a data object, for instance a look-up table, with 3030 entries and/or obtained via a network.

According to some preferred embodiments, variants or further developments of embodiments, the method according to the third or fourth aspect further comprises the steps of:
Providing correction data from or based on the individual identification code, IIC, light source and spectrum information; and
Saving the provided correction data, wherein the generation of the image data generated by the camera is adapted based on the saved correction data.

According to some preferred embodiments, variants or further developments of embodiments, the method according to the third or fourth aspect further comprises the steps of:
Checking the individual identification codes, IIC, or TIC of the light source to see whether it is noted (or flagged) as known, and if this is not the case,
Providing the spectrum information of the light source with this IIC or the type of light source with this TIC,
Storing the provided spectrum information in the memory; and
Noting (or flagging) the individual identification code, IIC, or TIC of the light source as known.

According to a fifth aspect, the invention provides a computer program product that comprises executable program code that, when it is executed, is designed to perform the method according to an embodiment of the third or fourth aspect of the present invention.

According to a sixth aspect, the invention provides a computer-readable, non-volatile data storage medium that comprises executable program code that, when it is executed, is designed to perform the method according to an embodiment of the third or fourth aspect of the present invention.

According to a seventh aspect, the invention provides a data stream that comprises executable program code or is designed to generate executable program code, wherein the program code is designed, when it is executed, to perform the method according to an embodiment of the third or fourth aspect of the present invention.

The above embodiments and developments can be combined with each other as desired, if appropriate. Further possible embodiments, developments, and implementations of the invention also include combinations, which are not explicitly mentioned, of features of the invention described above or below with respect to the exemplary embodiments. In particular, a person skilled in the art will also add individual aspects as improvements or additions to the particular basic form of the present invention.

The present invention is explained in greater detail below with reference to the exemplary embodiments shown in the schematic figures. In the drawings:

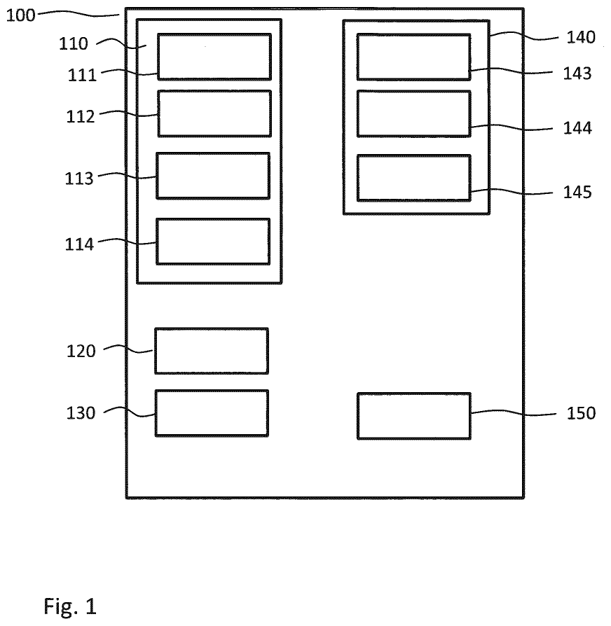

FIG. 1 is a schematic block diagram of a medical imaging system according to an embodiment of the present invention;

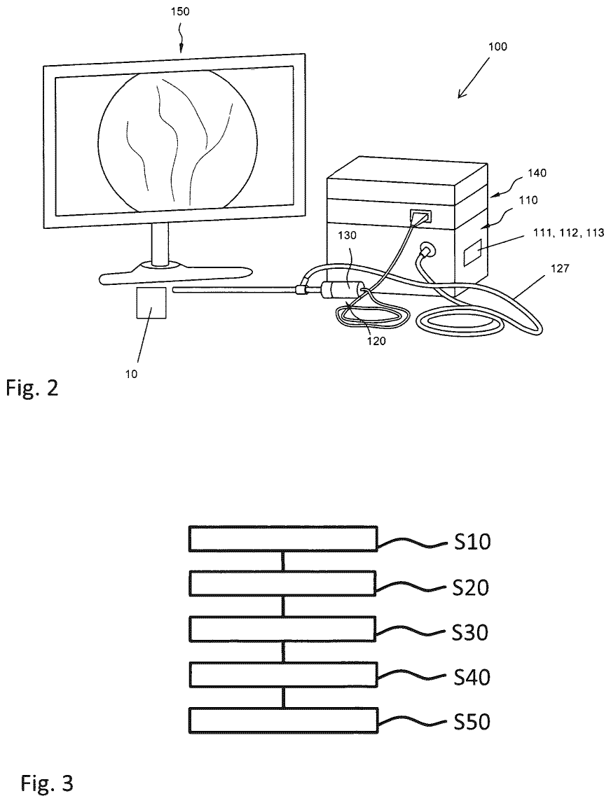

FIG. 2 is a schematic representation of a part of the medical imaging system of FIG. 1 according to a variant;

FIG. 3 is a schematic flow diagram for illustrating a method according to a further embodiment;

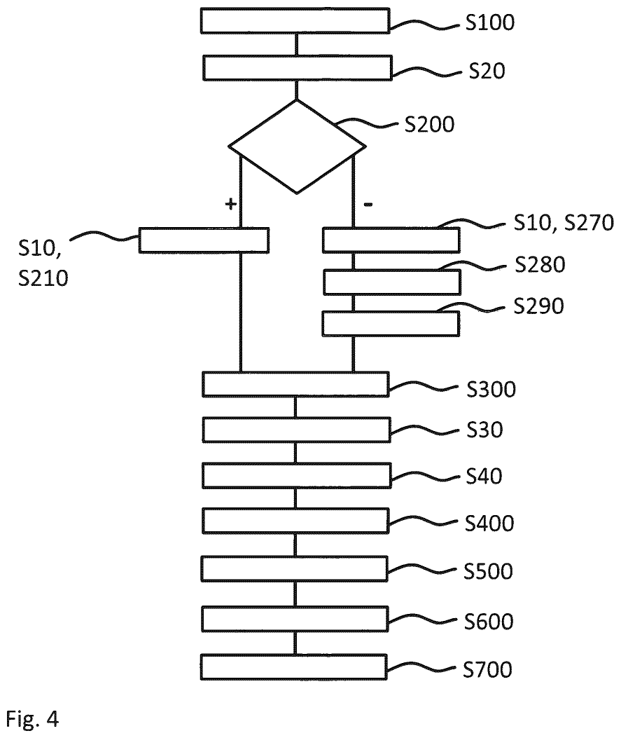

FIG. 4 is a schematic flow diagram for illustrating a method according to yet a further embodiment;

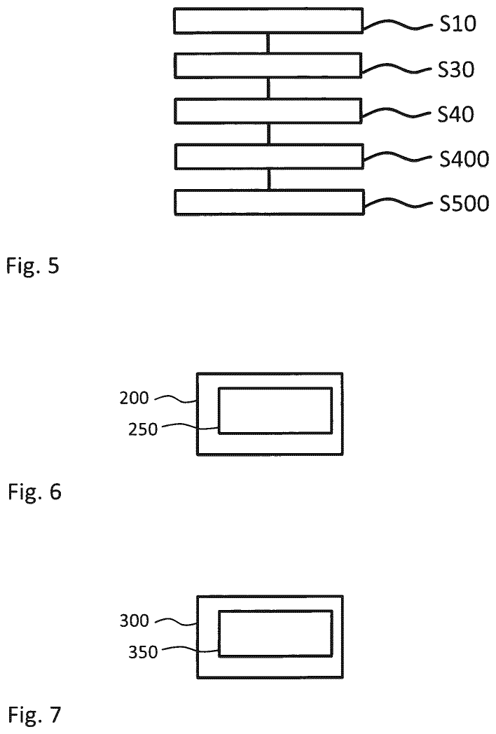

FIG. 5 is a schematic flow diagram for illustrating a method according to yet a further embodiment;

FIG. 6 is a schematic block diagram of a computer program product according to another embodiment of the present invention; and

FIG. 7 is a schematic block diagram of a data storage medium according to yet another embodiment of the present invention.

The accompanying figures are intended to provide a further understanding of the embodiments of the invention. They illustrate embodiments and, in conjunction with the description, serve to explain principles and concepts of the invention. Other embodiments and many of the advantages mentioned are shown in the drawings. The elements in the drawings are not necessarily shown to scale.

In the figures of the drawing, identical, functionally equivalent, and identically acting elements, features, and components are each provided with the same reference signs, unless otherwise specified. The numbering of method steps serves primarily to distinguish them and is not intended to necessarily imply a chronological order, unless explicitly stated otherwise.

### DESCRIPTION OF EXEMPLARY EMBODIMENTS

FIG. 1 shows a schematic block diagram of a medical imaging system 100 according to an embodiment of the present invention. In accompaniment thereto, FIG. 2 shows a schematic representation of a part of the medical imaging system 100 according to FIG. 1 according to a variant.

The medical imaging system 100 comprises a light source 110 to irradiate an object 10 with light of different wavelengths according to a current wavelength spectrum of a plurality of possible wavelength spectra of the light source 110. In the embodiment of FIG. 1, the light source 110 comprises a first memory 111 which is designed to store an individual identification code, IIC, of the light source 110 and in which such an IIC is also stored. The IIC can be, for instance, an alphanumeric code or a serial number.

Furthermore, the light source comprises a second memory 112 which is designed to store spectrum information concerning possible wavelength spectra of the light source 110 and in which such spectrum information is also stored, for example as a result of a series of tests after production of the light source.

The present invention is described with reference to FIG. 1 and FIG. 2 by means of an example in which a known change in the emitted wavelength spectrum due to an operating temperature dependence of the light source 110 is to be taken into account and compensated for. Accordingly, in this embodiment, the spectrum information can describe different temperature ranges, in each case having a width of between (or equal to) 1° C. to (or equal to) 5° C., and provide an associated wavelength spectrum of the light source 110 for each temperature range. This can be carried out, for instance, by means of a look-up table.

In the following, an alternative variant is assumed, according to which the spectrum information does not contain the complete spectral intensity spectra, but rather a look-up table with parameters by means of which evaluation algorithms can be adapted (correction data).

As already mentioned above, differently designated memories can also be implemented combined in one or more hardware storage devices. The first memory 111 and the second memory 112 can thus be implemented together in a hardware memory device, e.g., a solid-state memory. The data stored therein can also be implemented in a common data object, for instance by the individual identification code, IIC, occupying a defined field of the look-up table of the spectrum information.

As indicated in FIG. 2, the light of the light source 110 can be radiated directly or preferably indirectly, onto the object 10 according to the current wavelength spectrum. In FIG. 2, for instance, it is shown for example that the light from the light source 110 is guided via a light guide 127 to an endoscope 120 through which the light is then radiated onto the object 10. The medical imaging system 100 can thus also comprise an endoscope 120. However, exoscopic applications are also conceivable, so that the medical imaging system 100 can also comprise an exoscope. The object 10 can be located inside and/or outside a human body.

The medical imaging system 100 also comprises a camera 130, which is designed to generate image data of the object 10 with light reflected or emitted by the object 10 based on the light according to the current wavelength spectrum. As shown in FIG. 2, the camera 130 can be integrated into the endoscope 120. However, an exoscopic camera or the like is also conceivable.

The medical imaging system 100 also has a controller 140 which is designed to adapt, in particular correct, the image data generated by the camera 130 based on the individual identification code, IIC, the light source 110 and the spectrum information.

As shown in FIG. 2, the controller 140 can be integrated into a camera control unit (CCU) or can be formed as such, which typically also handles the image processing of the camera image. A screen device 150 for displaying the camera image can be an LCD screen, a touch screen or the like. In other variants, the controller 140 can also be integrated into the camera 130 and/or the endoscope 120.

The controller 140 is designed to obtain the individual identification code (IIC) of the light source 110 either regularly or in response to an event and to check whether it is noted as known. The event-based check can be performed, for example, as part of a handshake, i.e. in the event that the light source 110 is connected to the medical imaging system 100 (in particular the camera 130 and/or the controller 140). For this purpose, various interfaces can be used which can be configured as wired or wireless and can use different types of protocols, such as TCP/IP-based protocols. An example of this is the applicant's interface “KS Hive” (registered trademark). As already mentioned, the transmission of the IIC can be performed using the push method, using the pull method or according to other methods.

The fact that the individual identification code, IIC, is known to the controller 140 may mean, for example, that the IIC is stored in a memory 145 (sometimes referred to above as the “sixth memory”) of the controller 140.

If the IIC of the controller 140 is not known, the controller 140 is designed in the present example to obtain the spectrum information from the light source 110 from the second memory 112. In the present example, the spectrum information contains correction data, i.e., in this case parameters for an evaluation algorithm used by the controller 140, in each case depending on an operating temperature value of the light source 110.

These correction data are then stored, associated with the individual identification code, IIC, in a third memory 143 of the controller 140 and are then available if the light source 110 is coupled to this controller 140 again at a later point in time. Thus, if the same light source 110 is later coupled to the controller 140 again, only the IIC is checked, and only if there is a change (or if it is noted as unknown) is the spectrum information acquired and stored again.

In the present embodiment, the light source 110 further comprises a temperature sensor device 113 comprising at least one temperature sensor. By means of the temperature sensor device 113, a current operating temperature value of the light source 110 is captured and provided. If a plurality of temperature sensors are provided, they can capture individual temperatures at different locations. A value based on this can be output as the operating temperature value, for example an average value, a weighted average value, a median or the like.

The operating temperature value can be provided to the controller 140 regularly, in a push method, and/or in a pull method. By means of the correction data stored in the third memory 143, which are IIC-related, the controller 140 can thus optimally adapt the generation of the image data for the specific connected light source 110 with the known IIC and the known operating temperature value, in particular using a white balance or a color correction.

Depending on the wavelengths for which the light source 110, camera 130 and controller 140 are formed (visible, non-visible, . . . ), generating the image data can thus be adapted (or corrected) with respect to a large number of wavelengths. In particular, a multispectral image analysis (or a hyperspectral image analysis) can be performed.

The controller 140 can contain an evaluation module 144 which evaluates the image data generated in an adapted manner for the purpose of a medical diagnosis, for example, generates false color images that indicate various medical or physiological parameters such as oxygen saturation, hemoglobin content, water content, perfusion, and/or the like.

A visual representation of the image data in visible light and/or a false color image can be displayed, for example, by the screen device 150.

Data can be exchanged between the light source 110 and the controller 140 wired or wirelessly. A wired connection can be established via data lines that run indirectly via the endoscope 120, or can be made directly between the light source 110 and the controller 140.

In addition, or alternatively, to the temperature sensor device 113 and adapting the generation of the image data based on the current operating temperature value, the medical imaging system can also take into account the aging of the light source 110. For this purpose, an operating time or remaining operating service life of the light source 110 can be stored, for instance, in a memory 114 of the light source 110 (partially referred to above as the “fourth memory”) which is continuously updated by the light source 110 during its operation. In this case, the spectrum information can comprise information concerning a dependence of the wavelength spectrum of the light source 110 on the operating time or remaining operating service life of the light source 110. Obtaining and storing spectrum information and/or correction data along with adapting the generation of image data is then carried out analogously to that described above with reference to the operating temperature value.

A combination is also possible according to which, for a light source 110 (with a certain IIC), the spectrum information describes a dependence of the wavelength spectrum on both its current operating temperature value and its operating time/remaining operating service life, and adapting the generation of the image data is also carried out based on both.

It is particularly advantageous if the medical imaging system 100 is generally designed to obtain the spectrum information from light sources together with data relating thereto, if applicable from the light source 110, and to adapt the generation of the image data based thereon. For example, some light sources 110 can contain only IIC-dependent spectrum information, other light sources temperature-dependent spectrum information, other light sources 110 operating time-dependent spectrum information, other light sources 110 temperature-dependent and operating time-dependent spectrum information, and so on, wherein the controller 140 undertakes the best and most precise possible adaptation of the generation of the image data in each case.

With reference to FIG. 1 and FIG. 2, a medical imaging system 100, in particular for multispectral imaging, according to a further embodiment, in particular according to the third aspect of the present invention, can also be described. In so doing, differences compared to the previous embodiment are mainly explained; other elements can be implemented as previously described. The elements described below and the options provided for them can also be used in the previously described embodiment and vice versa.

The medical imaging system 100 explained below comprises, as already described above, a light source 110 that is designed to irradiate an object 10 with light of different wavelengths according to a current wavelength spectrum of the light source 110. In this embodiment, the case is described in which the current wavelength spectrum depends at least on the current operating temperature of the light source 110. The operating temperature may depend on the ambient temperature and/or (in particular) on the operating time of the light source 110. Thus, even given a constant ambient temperature, the operating temperature may change—in particular increase—over the time the light source 110 is put into operation, and thus the current wavelength spectrum of the light source 110 may also change.

Therefore, the medical imaging system 100, advantageously the light source 110, in this case comprises a memory 112 that is designed to store spectrum information concerning possible wavelength spectra depending on the operating temperature of the light source 110. As described in detail above, this can be done in absolute values, by means of adaptation data, by means of interpolations and/or the like. The content of the memory 112 can be based on the individual light source 110 and/or on the type or model series of the light source 110.

A temperature sensor device 113 of the light source 110 is designed to capture the (current) operating temperature value of the light source 110 during operation. As already explained with reference to FIG. 1, the operating temperature value can be a value calculated from a plurality of individual temperature measurement values, such as an average value or the like. Accordingly, the temperature sensor device 113 can also comprise one or more temperature sensors.

The medical imaging system 100 also comprises a camera 130 that is designed to generate image data of the object 10 with light reflected or emitted by the object 10 based on the light according to the current wavelength spectrum of the light source 110, as well as a controller 140 that is designed to adapt the generation of the image data by the camera 130 based on the spectrum information and the captured operating temperature value.

As explained above and below, this basic method can be implemented in many ways. For example, the controller 140 can be regularly or periodically informed by the light source 110 of the wavelength spectrum associated with the current operating temperature value (either directly, via adaptation data or the like). The controller 140 then can adapt the generation of the image data based on the wavelength spectrum.

In the variants in which spectrum information (or correction data obtained therefrom) that relate to the individual light source 110 or at least its type is stored within the controller 140, the method described above and below, particularly in connection with FIG. 4, can be used, according to which an individual identification code, IIC, of the light source 110 is used to determine whether a change of the light source 110 has taken place and, if so, whether an already known light source 110 has been connected, or a still unknown light source 110. Alternatively, for example, the method described with reference to FIG. 5 can also be performed.

Instead of an individual identification code, IIC, a type identification code, TIC, could also be used, which is used for all light sources 110 of the same type. This is advantageous if variations in the wavelength spectra within a type—e.g., due to manufacturing—are negligible compared to the effects of the operating temperature value.

Whether or not the light source 110 is known can be established, for example, by whether the memory 143 of the controller 140 contains spectrum information and/or correction data of the light source 110. Alternatively, there may also be a further memory 145 (partially referred to above as the “sixth memory”) of the controller 140, which contains a list of the IICs of all known light sources 110.

If the light source 110 is unchanged, the spectrum information currently used by the controller 140 is still correct. If the light source 110 has been newly connected but is already noted as known, the associated spectrum information (or correction data obtained therefrom) can advantageously be read out from data stored in the controller 140.

If a new light source 110 that has not yet been noted as known has been connected, the spectrum information can be automatically read out from a memory 112 of the light source 110 itself (or from another memory, e.g., a cloud memory) and copied, for example, into the controller 140 (e.g., into the memory 143), for instance as part of a handshake. This light source 110 (more precisely: its individual identification code, IIC, or its type identification code, TIC) is then noted as known.

It is also possible that the light source 110 in each case transmits only information concerning the current operating temperature value to the controller 140. For this purpose, the spectrum information can be stored, for example, in the memory 143 of the controller 140, wherein the controller 140 then reads out the corresponding spectrum information based on the received current operating temperature value. It is also conceivable that the spectrum information comprises operating temperature-dependent correction data and that the correction data read out from the spectrum information (e.g., parameters of an algorithm) of the light source 110 in each case currently connected to the controller 140 are or will be stored in the memory 143, which are then used by the controller 140 to adapt the generation of the image data.

FIG. 3 shows a schematic flow diagram for explaining a method according to an embodiment of the third aspect of the present invention, i.e. a method for operating a medical imaging system. This method can be performed in particular using the medical imaging system 100 according to FIGS. 1 and 2 (or is applicable for operating this imaging system 100), and vice versa. Therefore, repetitions will be largely avoided.

In a step S10, spectrum information concerning possible wavelength spectra of a light source 110 is provided to the medical imaging system 100, e.g., in a memory 112 of the light source 110 as previously described.

This can be carried out, for example, in the broadest sense by measuring, as already explained above, light intensities of the light emitted by the individual light source 110 with the individual identification code, IIC, in a steady-state condition, depending on different operating temperatures. For example, the light intensities can be measured between (inclusive) 400 nanometers wavelength and 1200 nanometers wavelength in steps of 5 nanometers. Based on this, operating temperature-dependent spectrum information can be created.

In a narrower sense, the spectrum information can also be provided by the second memory 112 itself.

In a step S20, an individual identification code, IIC, of the light source 110 is provided to the medical imaging system 100, or specifically to a controller 140 of the medical imaging system 100, for example in the previously described first memory 111 of the light source 110.

In a step S30, an object 10 is irradiated by the light source 110 with light of different wavelengths according to a current wavelength spectrum of the light source, in particular as described above with reference to the light source 110.

In a step S40, image data of the object 10 are generated with light reflected or emitted by the object 10, in particular by means of absorption spectroscopy, based on the light according to the current wavelength spectrum. This can be carried out, for example, as described above with reference to the camera 130 and/or the controller 140.

In a step S50, the generation S40 of the image data is adapted, in particular corrected, based on the individual identification code, IIC, of the light source 110 (or using the individual identification code, IIC, of the light source 110 and the spectrum information. This can also be carried out as described above with reference to the controller 140.

FIG. 4 shows a schematic flow diagram for explaining a method according to a further embodiment. The method according to FIG. 4 represents a special variant of the method according to FIG. 3, which is adapted analogously to the medical imaging system 100 from FIG. 1 and FIG. 2.

In FIG. 4, in a step S100, a connection is established between a light source 110 and a controller 140 (also called a camera control unit or “CCU”). This connection can be wired or wireless as described above.

Then, as part of a handshake, the provision S20 of a serial number (or another individual identification code, IIC) of the light source 110 is carried out from the light source 110 to the controller 140. The IIC is preferably stored in a memory 111 of the light source 110.

In a step S200, using the IIC (here: the serial number), it is checked whether the light source 110 is noted as known to the controller 140. In this exemplary embodiment, this is equivalent to the controller 140 having spectrum information concerning this specific light source 110.

As will become apparent below, this is at least the case if this specific light source 110 and this specific controller 140 have already been connected to one another as part of a medical imaging system 100 according to the invention.

If the result of the check in step S200 is positive (“+” in FIG. 4), in a step S210, the controller 140 provides the available spectrum information for the light source 110 based on its IIC for further use, for example, read from a memory 143 of the controller 140 into a working memory of the controller 140.

If the result of the check in step S200 is negative (“−” in FIG. 4), in a step S270, the controller 140 obtains the spectrum information for the light source 110 based on its IIC, for example read out by the controller 140 from a memory 112 of the light source 110, read out from a cloud-based database based on the IIC and/or the like. In a step S280, the spectrum information can then also be stored in the memory 143 of the controller 140, and in a step S290, the IIC of this light source 110 can be noted as known, e.g., using the memory 145.

Thus, the provision S10 of the spectrum information is carried out, in particular, to the controller 140 either by step S210 or by step S270.

In a step S300, imaging, in particular multispectral imaging for absorption spectroscopy, can then be started, for example in a human body. The step S300 can be triggered by a user, e.g., by a user interface connected to the controller 140, the camera 130 and/or the light source 110, or can be carried out automatically. The step S300 can comprise starting the recording by the camera 130, beginning to irradiate the object 10 with light from the light source 110, etc. Accordingly, the steps S30 and S40 are then performed as described above.

In a step S400, a current operating temperature value is captured, in particular by a temperature sensor device 113 of the light source 110, and transmitted to the controller 140. This is carried out in particular during irradiation S30 and generation S40. In this way, the utilized correlation between the operating temperature value and the lighting situation during the generation S40 of the image data is advantageously particularly close.

In a step S500, the controller 140 adapts, e.g., corrects, the generation S40 of the image data based on the provided spectrum information and based on the captured operating temperature value. As already mentioned, the spectrum information for this purpose can have operating temperature-dependent wavelength spectra, operating temperature-dependent correction data and/or the like.

The steps S400 and S500 in FIG. 4 can be performed regularly (or continuously). They can also be applied analogously to other time-dependent properties of the particular light source (e.g., aging phenomena).

Subsequently, further steps may follow, for example an evaluation S600 of the image data generated in an adapted manner (as already described above, for example in connection with the evaluation module 144), and/or a visual representation S700 of the image data generated in an adapted manner in visible light and/or a false color image (as already described above, for example in connection with the screen device 150).

In the embodiment described with reference to FIG. 4 as well, the individual identification code, IIC, in each case can be replaced by a type identification code, TIC.

FIG. 5 serves to schematically represent a method according to a further embodiment of the present invention, in connection with the fourth aspect.

In step S10, spectrum information concerning possible wavelength spectra of a light source 110 is provided to the medical imaging system 100, e.g., in a memory 112 of the light source 110 as described above, wherein the spectrum information in this case describes (or takes into account) in particular (or at least) a dependence of the wavelength spectra on the operating temperature value of the light source 110.

In the step S30, an object 10 is irradiated by the light source 110 with light of different wavelengths according to a current wavelength spectrum of the light source, wherein the current wavelength spectrum results in particular directly from the current operating temperature value of the light source 110.

In step S40, the image data of the object 10 are generated with light reflected or emitted by the object 10, in particular by means of absorption spectroscopy, based on the light according to the current wavelength spectrum. This can be carried out, for example, as described above with reference to the camera 130 and/or the controller 140.

In step S400, the current operating temperature value is captured, in particular by a temperature sensor device 113 of the light source 110, and transmitted to the controller 140.

In step S500, the controller 140 adapts, e.g., corrects, the generation S40 of the image data based on the provided spectrum information and based on the captured operating temperature value.

It is understood that the optional steps S600 and S700 of FIG. 4 can also be performed in the methods according to FIG. 3 or FIG. 5.

FIG. 6 shows a schematic block diagram of a computer program product 200 according to an embodiment of the present invention. The computer program product 200 comprises executable program code 250 that, when it is executed, is designed to perform the method according to an embodiment of the present invention, for example according to FIG. 3 or FIG. 4.

FIG. 7 shows a schematic block diagram of a non-volatile computer-readable data storage medium 300 according to an embodiment of the present invention. The data storage medium 300 comprises executable program code 350 that, when it is executed, is designed to perform the method according to an embodiment of the present invention, for example according to FIG. 3 or FIG. 4.

The non-volatile computer-readable data storage medium 300 can, for example, be formed as or have a semiconductor memory, e.g., an SSD memory chip. The data storage medium 300 can also comprise or consist of a CD, DVD, Blu-ray or a magnetic storage device.

In the previous detailed description, various features have been summarized in one or more examples to improve the stringency of the presentation. It should be understood, however, that the above description is merely illustrative and not restrictive. It is intended to cover all alternatives, modifications and equivalents of the various features and exemplary embodiments. Many other examples will be immediately and directly clear to a person skilled in the art on the basis of their technical knowledge in view of the above description. The exemplary embodiments were selected and described in order to best illustrate the principles underlying the invention and its possible applications in practice. This allows for those skilled in the art to optimally modify and utilize the invention and its various exemplary embodiments in relation to the intended purpose.

### LIST OF REFERENCE SIGNS

10 Object
100 Medical imaging system
110 Light source
111 First memory
112 Second memory
113 Temperature sensor device
114 Fourth memory
120 Endoscope
127 Light guide
130 Camera
140 Controller
143 Third memory
144 Evaluation module
145 Sixth memory
150 Screen device
200 Computer program product
250 Program code
300 Data storage medium
350 Program code
S10 . . . . S50 Method steps
S100 . . . . S700 Method steps

1. A medical imaging system, in particular for multispectral imaging,
comprising a light source which is designed to irradiate an object with light of different wavelengths, according to a current wavelength spectrum of the light source, wherein the light source is assigned an individual identification code, IIC,
a first memory which is designed to store the individual identification code, IIC, of the light source,
a second memory, which is designed to store spectrum information concerning possible wavelength spectra of the light source,
a camera which is designed to generate image data of the object with light reflected or emitted by the object, based on the light according to the current wavelength spectrum of the light source, and
a controller which is designed to adapt the generation of image data by the camera using the individual identification code, IIC, of the light source and the spectrum information.

2. The medical imaging system according to claim 1, wherein the controller is designed to provide correction data from or based on the individual identification code, IIC, of the light source and the spectrum information, wherein the medical imaging system further comprises a third memory which is designed to store the correction data, and wherein the controller is designed to adapt the generation of the image data based on the stored correction data.

3. The medical imaging system according to claim 1, comprising a temperature sensor device which is designed to capture an operating temperature value of the light source during operation and to transmit the captured operating temperature value to the controller; wherein the controller is designed to additionally adapt the generation of the image data based on the operating temperature value.

4. The medical imaging system according to claim 3, wherein the spectrum information comprises information that, for the light source having the individual identification code, IIC, in each case allocates a wavelength spectrum of the light source to a plurality of operating temperature values, and wherein the controller is designed to select a wavelength spectrum assigned to the captured operating temperature value based on the spectrum information and to adapt the generation of the image data based on the selected wavelength spectrum.

5. The medical imaging system according to claim 4, wherein the temperature sensor device is designed to capture the operating temperature value of the light source on request and/or at predetermined time intervals and to communicate this to the controller, wherein the controller checks whether the captured operating temperature value is assigned to the selected wavelength spectrum and, in the event of an unsuitable assignment due to a change in the operating temperature value, selects a different wavelength spectrum that is assigned to the captured operating temperature value.

6. The medical imaging system according to claim 1, wherein the controller is designed to check the individual identification code, IIC, of the light source as to whether it is noted as known and, if this is not the case, to obtain the spectrum information of the light source, store it in a memory and note the individual identification code, IIC, of the light source as known.

7. The medical imaging system according to claim 1, wherein adapting the generation of the image data comprises a white balance.

8. The medical imaging system according to claim 1, wherein adapting the generation of the image data comprises color correction.

9. The medical imaging system according to claim 1, wherein the camera is designed to record spectra, and the image data comprise spectral data.

10. The medical imaging system according to claim 1, wherein the light source is designed to irradiate the object with light in visible wavelengths and in at least one invisible wavelength.

11. The medical imaging system according to claim 1, wherein the camera is designed to generate image data of light in visible and invisible wavelengths.

12. The medical imaging system according to claim 1, having an evaluation module which is designed to evaluate the generated image data for the purpose of determining physiological tissue properties.

13. The method for operating a medical imaging system, in particular according to claim 1, having the steps of:
providing spectrum information concerning possible wavelength spectra of a light source;
providing an individual identification code, IIC, of the light source;
irradiating, by the light source, an object with light of different wavelengths according to a current wavelength spectrum of the light source;
generating image data of the object with light reflected or emitted by the object based on the light according to the current wavelength spectrum, wherein generating the image data is adapted using the individual identification code, IIC, of the light source and the spectrum information.

14. The method according to claim 13, comprising the further step of:
capturing an operating temperature value of the light source, wherein adapting the generation of the image data is carried out based, among other things, on the captured operating temperature value.

15. The method according to claim 13, comprising the steps of:
checking the individual identification code, IIC, of the light source as to whether it is noted as known to a controller, and if this is not the case,
providing the spectrum information of the light source with this individual identification code, IIC, to the controller,
storing the provided spectrum information in a memory of the controller; and
noting the IIC of the light source as known to the controller.

16. The computer program product comprising executable program code that, when it is executed, is designed to perform the method according to claim 13.

17. The computer-readable non-volatile data storage medium comprising executable program code that, when it is executed, is designed to perform the method according to claim 13.

18. The data stream that comprises executable program code or is designed to generate executable program code, wherein the program code is designed, when it is executed, to perform the method according to claim 13.
